(19) United States
(12) Patent Application Publication
(10) Pub. No.: US 2006/0142665 A1
(43) Pub. Date: Jun. 29, 2006

(54) Heart rate monitor

(76) Inventors: John L. Garay, Southbury, CT (US); Louis M. Galie, Newtown, CT (US); Dan Simoneau, Bend, OR (US); Wilson W. Keithline, New Britain, CT (US)

Correspondence Address: Arthur G. Schaier;Carmody & Torrance LLP, 50 Leavenworth Street, P.O. Box 1110, Waterbury, CT, 06721-1110, US

(21) Appl. No.: 10/846,441

(22) Filed: May 14, 2004

(51) Int. Cl. A61B 5/02

(52) U.S. Cl. 600/508

(57) ABSTRACT

A wearable fitness heart rate monitor comprising an earpiece comprising a sensor unit having a light emitting element and a light receiving element, wherein a portion of an ear is positioned therebetween, wherein the light emitting element emits light into the portion of the ear positioned intermediate the light emitting and light receiving elements, the light receiving element detects the intensity of light passing through the portion of the ear positioned intermediate the light emitting and light receiving elements, and the sensor unit comprises means for outputting an electrical signal based thereon; processor means, coupled to the sensor unit, for processing the electrical signal and determining a heart rate therefrom; and a display, coupled to the processor means, for displaying the heart rate. Alternatively, the transmitting element (LED) and the receiving element (photodiode or the like), are positioned adjacent each other, such that the transmitting element emits light into the portion of the ear and the receiving element receives reflected light therefrom, and wherein the sensor unit detects the intensity of the reflected light and outputs electrical signals based thereon.

### BACKGROUND OF THE INVENTION

The present invention relates generally to heart rate monitors, and in particular, to a heart rate monitor that detects one's heart rate from a sensor located about the ear so that a least a portion of the heart rate monitor, namely, at least the portion via which the pulse rate is detected, may be worn about the head, such as in the form of a headset.

Heart rate monitors are well known, being used in a variety of fields of endeavor, including the medical and fitness fields. In the medical field, heart rates may be monitored in a variety of ways, most of which are prohibitive in the area of the “fitness world” due to the physical nature of athletic activities.

That is, in the “fitness world,” popular heart rate monitors typically come in the form of a two-piece “system” comprising a cheststrap, which (at least) detects the heartbeat, and a wrist worn unit, which is typically used to display a calculated heart rate. Such designs are widely available by a number of manufacturers, including Timex Corporation, Polar, Nike, just to name a few. There are also many patents describing such configurations.

The inventors of the present invention are also aware of wristwatch-type heart rate monitors which detect the pulse wave using sensors about the finger, such as that which is described in U.S. Pat. Nos. 6,198,951; 5,152,296; 4,938,228; 4,425,921; 4,412,546 and 4,353,152. However, it is perceived that there are disadvantages of using the finger as a source of the pulse wave detection, such as unreliability of results via inaccurate pulse wave detections, and discomfort during vigorous exercise. Similar discomfort is a perceived disadvantage in using the cheststrap unit as well.

The prior art also recognizes the desirability of detecting a heart rate from the inner ear, two such descriptions being set forth in U.S. Patent Application Publication No. 2002/0091049 and Japanese Publication No. 07-241279. However, in patentable distinction to that which will be hereinafter described, these aforementioned publications describe the method whereby the temporal artery is irradiated with light.

Although the inventors of the present invention believe that the foregoing particular construction is likewise less than desirable, the inventors do believe that utilizing the ear region to detect the pulse rate is advantageous. Specifically, the present inventors believe that utilizing a sensor to enclose or otherwise “sandwich” a portion of the ear, such as the earlobe (by way of example and not limitation) is desirable for several reasons, such as the ease by which it can be worn, the reduction of noise artifacts during high levels of physical activity, and the ease by which such an arrangement can be integrated into a headset, such as a visor or other eyewear assembly. The present invention also provides for improved stand-alone displaying of information and/or communication with a wristworn device and/or a personal computer. As such, the present inventors disclose an embodiment that others have discouraged (see the aforementioned Published Application No. 2002/0091049 at paragraph [0030]).

Accordingly, it is desirable to provide a heart rate monitor with an accurate and comfortable, convenient heartbeat detector that overcomes the perceived deficiencies in the prior art noted above and further achieves the aforementioned and below mentioned objectives.

### SUMMARY AND OBJECTIVES OF THE INVENTION

Accordingly, it is an objective of the present invention to provide a heart rate monitor that overcomes the perceived deficiencies in the prior art.

Another objective of the present invention is to provide a heart rate monitor that is comfortable to wear, and which provides accurate heart rate information.

Another object of the present invention to provide a heart rate monitor that can be worn about a user's head, such as in a heart rate monitor that incorporates a display that may be provided in the headset itself.

Yet another objective of the present invention to provide a heart rate monitor that can be used in combination with a wrist worn display unit or an eyewear display unit.

Still another objective of the present invention to provide a heart rate monitor that can store information for later review and/or playback, such as on a stand-alone computer, as but one example.

Still other objects and advantages of the invention will in part be obvious and will in part be apparent from the specification.

The invention accordingly comprises the features of construction, combination of elements, arrangement of parts and sequence of steps which will be exemplified in the construction, illustration and description hereinafter set forth, and the scope of the invention will be indicated in the claims.

Generally speaking, in accordance with the present invention, an improved heart rate monitor is provided. In a preferred embodiment, the wearable fitness heart rate monitor comprises an earpiece comprising a sensor unit having a light emitting element and a light receiving element, wherein a portion of an ear is positioned therebetween, wherein the light emitting element emits light into the portion of the ear positioned intermediate the light emitting and light receiving elements, the light receiving element detects the intensity of light passing through the portion of the ear positioned intermediate the light emitting and light receiving elements, and the sensor unit comprises means for outputting an electrical signal based thereon; processor means, coupled to the sensor unit, for processing the electrical signal and determining a heart rate therefrom; and a display, coupled to the processor means, for displaying the heart rate.

In specific implementations, the heart rate monitor is incorporated into a headset, to which the earpiece is coupled, wherein the display is mechanically coupled to the headset and provided on a visor. In another implementation, an eyewear unit is provided with the display for displaying the heart rate determined by the processor means. Alternatively, a wrist worn unit may be provided with the display for displaying the heart rate determined by the processor means. All of the foregoing communication may also be performed wirelessly to the display. A system for playing back stored heart rate information is also provided, and may comprise a computer coupleable to the heart rate monitor, wherein the computer comprises means for displaying the information downloaded from the means for storing information.

In yet another embodiment, the wearable fitness heart rate monitor comprises an earpiece comprising a sensor unit having a transmitting element and a receiving element, wherein the transmitting element and the receiving element are positioned adjacent each other, wherein the sensor unit is placed proximate to a portion of an ear, wherein the transmitting element emits light into the portion of the ear positioned proximate thereto and the receiving element receives reflected light therefrom, and wherein the sensor unit detects the intensity of the reflected light and outputs electrical signals based thereon; processor means for processing the electrical signals and determining a heart rate therefrom; and a display, coupled to the processor means, for displaying the heart rate.

### BRIEF DESCRIPTION OF THE DRAWINGS

For a fuller understanding of the invention, reference is had to the following description taken in connection with the accompanying figures, in which:

FIG. 1 is a perspective view illustrating a heart rate monitor and system, constructed in accordance with a first embodiment of the present invention;

FIG. 2 is a plan view of an earpiece taken about lines 2-2 in FIG. 1;

FIG. 3 is a plan view of an alternative construction of the heart rate monitor of FIG. 1, merely illustrating differing head strap configurations;

FIG. 4 is a simplified block diagram of hardware configured in accordance with and used in connection with the present invention;

FIG. 5 is a plan view of an earpiece constructed in accordance with an alternative embodiment; and

FIG. 6 is a simplified perspective view of an alternative embodiment of the present invention, utilizing eyewear, such as eyeglasses, which comprise the display.

Also, while not all elements are labeled in each figure, all elements with the same reference number indicate similar or identical parts.

### DETAILED DESCRIPTION OF THE PREFERRED EMBODIMENTS

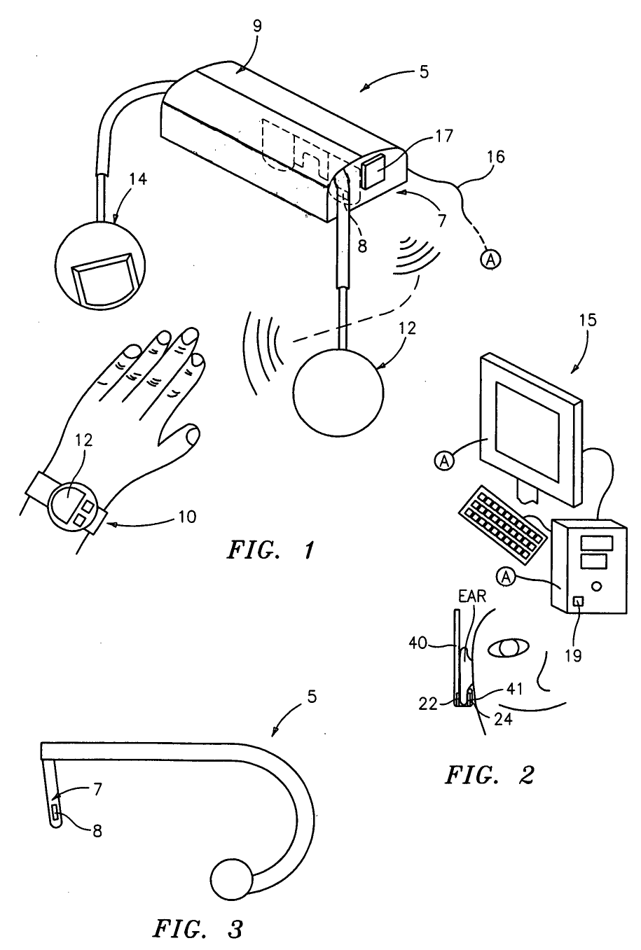

Reference shall first be made to FIGS. 1-3 for an overall disclosure of the heart rate monitor and system constructed in accordance with the present invention. Specifically, comprising the wearable heart rate monitor of the present invention is a headset unit, generally indicated at 5. As will be more fully appreciated below, the heart rate monitor of the present invention (hereinafter the “HRM” for ease of reading) comprises a display for displaying the heart rate to a user.

Three types of wearable displays are contemplated herein and are in accordance with the preferred embodiments. The first embodiment comprises a visor with a built-in display, such as LCD. Such an exemplary construction, generally indicated at 7 and including a display unit 8, is illustrated in FIG. 1 as being depending from a headpiece unit, generally indicated at 9. A similar display unit 8 is shown in FIG. 3, which itself illustrates another design configuration for carrying the circuitry and wiring from the sensor to the display and/or for transmitting signals to a wrist worn display as further disclosed below. For purposes of clarity, it should be understood that the HRM of FIG. 1 need not have a visor/display construction 7, since a different type of display, such as a wrist worn display 12 may be used in place thereof. That is, it is envisioned that visor display 7, such as that indicated by display 8, is optionally provided. Accordingly, the particular illustration thereof in FIG. 1 should be understood to be in the exemplary and not limiting sense. That is, either display (and not necessarily both) will fall within the scope of the claims.

If a visor display 7 is desired, a miniaturized and/or retractable construction is preferred. Constructing such a visor using retractable and flexible supports and wires, and an LCD that is sufficiently small enough, along with conventional electronics and mechanical assemblies to construct the visor assembly 7, is believed to be within the scope of the skilled artisan.

In an alternative arrangement and illustrated in FIG. 6, the present invention contemplates use of eyewear, generally indicated at 7′, such as glasses (e.g. with and without prescription lenses (e.g. protective lens)), that are physically disconnectable from the earpiece. In specific embodiments, the eyewear would be physically disconnected from headset unit 5 if wireless communication (e.g. RF) is used to transmit the heart rate information to the eyewear unit 7′ but could be physically connected thereto in the event that a link, such as a “wired link” (of which a USB is but one example), were utilized. Similarly, processors could be used as needed to process the information being communicated between the headset unit 5 and the eyewear unit 7′. In a preferred embodiment, eyewear unit 7′ includes the LCD 8 for displaying of the heart rate or other information, and may be attached to or embedded therein.

On the other hand, the display for displaying the heartbeat may be provided on a wrist worn unit, generally indicated at 10, with the display particularly indicated at 12. It is believed that it is well within the purview of one skilled in the art to wirelessly (or in a hardwire fashion if desirable) transmit signals from headset unit 5 to wrist worn unit 10, which would then be appropriately processed for display as understandable information (i.e. the heart rate) to a user. To be sure, the wristworn unit may be a digital wristwatch or an analog wristwatch, such as those exemplary illustrated in U.S. patent application Ser. No. 10/441,417, the disclosure of which is incorporated by reference as if fully set forth herein. For example, a general description can be found in coowned and copending application Ser. No. 10/627,093, the disclosure of which is incorporated herein by reference for it description relating to the methodology of computing sampled heart rate values and then transmitting the computed heart rate values to the accompanying wrist worn device and/or computing the sampled heart rate values in the wrist worn device watch only after data representative of the sampled heart rate is taken and transmitted to the wrist worn device. Likewise, the disclosures of U.S. Pat. Nos. 6,026,335; 6,345,197 and 4,566,461, to the extent they describe how a heartbeat is measured or sampled, how a heart rate value is computed and/or how information relating to the heart rate is displayed on a device (i.e. a piece of exercise equipment) and/or transmitted to another device, such as a wrist worn device, and how such a heart rate is displayed or conveyed to a user, along with the design, construction and implementation of an alarm (audible and/or visible), are all incorporated by reference as if fully set forth herein. For the same reason, the disclosure of U.S. Pat. No. 6,332,094 is also incorporated by reference as if fully set forth herein for its similar descriptions.

Therefore, and notwithstanding the fact that one skilled in the art could, from the aforementioned disclosures which are incorporated by reference, adequately and easily design and construct a heart rate monitor that could utilize and implement the present invention, a few specific details of the preferred transmission protocol is set forth. Specifically, in the preferred construction, the methodology of the present invention is incorporated into a heart rate monitoring system comprising (i) a transmitter located at or about the earpiece, generally indicated at 14, or in the headpiece unit 9. For convenience and not limitation, a transmitter that transmits signals from headset unit 9 to a wrist worn or other wirelessly coupled display, is generally indicated at 17, and is applicable to all embodiments set forth herein. Alternatively, a physical connection (e.g. a “wired link”) could be made between headset unit 9 and any of the display units (e.g. wristworn unit 10). In the preferred method, transmitter 17 transmits a packet of data, comprising among other things the computed sampled heart rate value, to the device, such as glasses 7′ or wrist worn device 10 (which itself would comprise a corresponding receiver 18), in predetermined time intervals, such as by way of example and not limitation, every two seconds. Thus, it is clear that the transmission rate may be independent of the heart rate that the transmitter is measuring. As would be understood in the art, after receiver 18 receives the packet of data, wrist worn unit 10 processes the information and displays the heart rate values on display 8 or 12. Likewise, there could be communication from the watch to the personal computer as disclosed below, such as compatible transmitting/receiving “dongles” that incorporate both/either RF or “wired link” (of which a USB link is but one example) circuitry and interface with one another. The headset unit could be similarly configured to interface therewith.

That is, in accordance with the present invention, a heart rate monitoring system is provided. In the preferred embodiment, the system comprises headset unit 5 and an external, preferably stand-alone computer, such as that illustrated and indicated by reference numeral 15. An external wire 16 (e.g. a “wired link” of which a USB is but one example) may be used to couple headset unit 5 with external computer 15, or headset unit 5 may be wirelessly linked to computer 15 through other conventional means, such as transmitter stage 17 (in headset unit 5) and a receiver unit 19 in the computer 15. Communication is preferably via an RF signal, all of which should be well understood by the skilled artisan. In such an embodiment, it should be understood that the heart rate monitor of the present invention preferably comprises means for storing information relating to the heart rate, such as in the form of volatile RAM, and means for downloading the information to the stand-alone computer unit, such as via the RF transmitter/receiver pair or other conventional circuitry (e.g. a “wired link” of which a USB is but one example). In a similar manner, computer 15 will preferably comprise means for displaying the information downloaded from the headset unit 15, all of which and more (e.g. combining all of the foregoing with a GPS unit), should be understood in the art, although details of which may be found in U.S. Pat. Nos. 6,002,982; 6,148,262 and 6,463,385, the disclosures of which are incorporated by reference as if fully set forth herein.

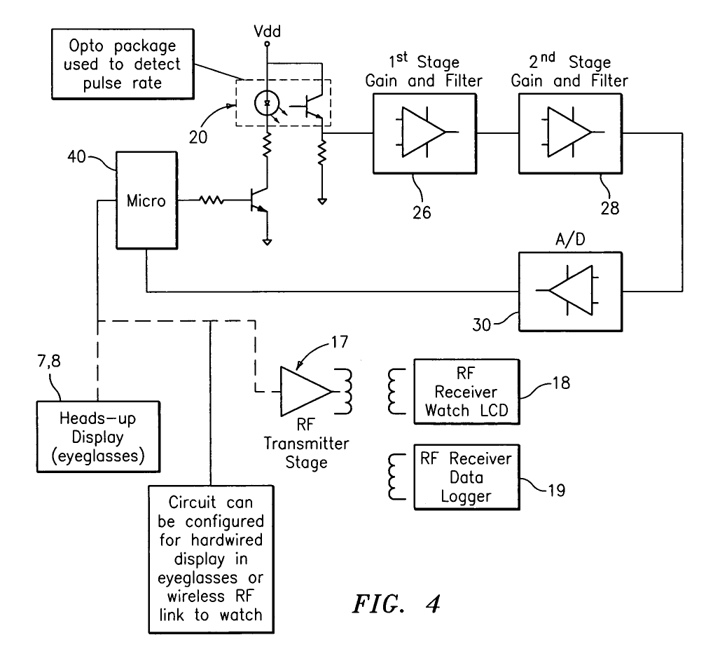

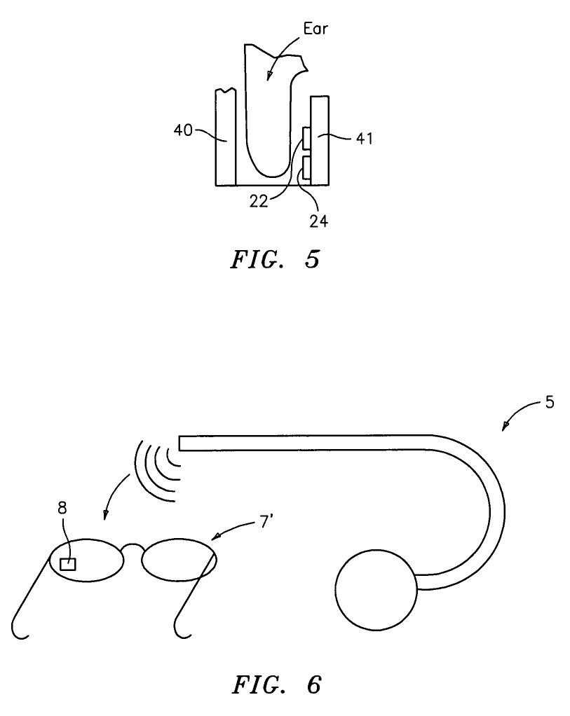

Reference is now made to FIGS. 2 and 5 for disclosure of preferred embodiments of sensor units, and FIG. 4 for a disclosure of circuitry for emitting light into the portion of the ear, for detecting the intensity of light either reflected by or passing through the portion of the ear positioned intermediate the opposing (or adjacent, as illustrated in FIG. 5) elements, and for outputting an electrical signal based thereon.

As first illustrated in FIG. 2, earpiece 12 comprises a sensor unit, generally illustrated in FIG. 4 by reference numeral 20, comprising a light-emitting diode (“LED”) 22 and one or more photodiodes 24 opposing and in facing alignment with LED 22. These components are preferably mounted on a circuit board, with the appropriate wiring and power being supplied thereto. FIG. 2 illustrates a portion of the ear, preferably but not necessarily the earlobe, “sandwiched” (e.g. disposed) between opposing elements 22 and 24. Supporting members 40, 41 on which the respective elements 22 and 24 are mounted, may be formed of plastic similar to a conventional pair of headphones. When LED 22 emits light to the ear, emitted light is absorbed by hemoglobin in the blood flowing through blood capillaries in the tissues, light that is not absorbed passes through the tissue, and the “passed through” light is received by the photodiodes 24 and converted to an electrical signal according to the amount of received light. Preferably, the earpiece itself does not pass light so that even when the present invention is used outdoors, natural light will not be directly incident on photodiodes 24.

Alternatively, as illustrated in FIG. 5, the transmitting (e.g. LED) element 22 and the receiving (e.g. photodiode(s)) element 24 are mounted on one side of the earlobe (either the inner side nearer the neck of the person or on the outer side of the lobe), and are thus positioned adjacent each other, for example, positioned horizontally and/or vertically with respect to each other (FIG. 5 shows elements 22, 24 positioned vertically but this is by way of example and not limitation). In this embedment, LED 22 and the phototransistor/photodiode 24 are mounted in the same plane, with the amount of light reflected by the tissue when blood is pumped through the artery being detectable by phototransistor or photodiode 24.

Sensor 20 will convert a biosignal mixed with noise into a corresponding electric signal. Sensor 20 is electrically connected to an amplifier arrangement, which in the preferred embodiment, comprises amplifiers 26 and 28 for appropriate amplification and filtering, as would be understood by one skilled in the art. The filtering performed by amplifier 26 limits the frequency components of the biosignal mixed with noise so as to satisfy the required sampling criterion when these signals are later digitized. The filtering also band limits low frequency noise. The corresponding filtered signal may then be amplified again by amplifier 28. The signal is then outputted to a circuit 30, which in the preferred embodiment may be a comparator or an analog to digital converter (A/D). The output from circuit 30 is the resultant preprocessed signal that is input to processor 40. Preferably, the circuitry of FIG. 4 is located in the headpiece unit 9 of headset 5.

In the preferred embodiment, the preprocessed signal is processed by processor 40 in order to determine the user's heart rate. Digital pulse processor 40 preferably also includes a microprocessor, which are both conventional and readily available, such as by Texas Instruments, Motorola, Epson or Microchip. Again the remainder of the details would be understood by one skilled in the art, and in fact most particularly set forth in the aforementioned U.S. Pat. No. 4,938,228.

It can thus be seen that the present invention provides numerous advantages not found in the prior art. For example, the present invention provides a heart rate monitor that is comfortable to wear, and which provides accurate heart rate information. The present invention also provides an embodiment that incorporates a display that may be provided in the headset itself. The present invention also provides an embodiment that can be used in combination with the more traditional wrist worn display unit or an eyewear display unit. Also the present invention can be integrated with other features, such as earpieces that can convey music through miniature speakers that do not get in the way of the pulse wave detections.

While the invention has been particularly shown and described with respect to preferred embodiments thereof, it will be understood by those skilled in the art that changes in form and details may be made therein without departing from the scope and spirit of the invention.

What is claimed is:

1. A wearable fitness heart rate monitor comprising:
an earpiece comprising a sensor unit having a light emitting element and a light receiving element, wherein a portion of an ear is positioned therebetween, wherein the light emitting element emits light into the portion of the ear positioned intermediate the light emitting and light receiving elements, the light receiving element detects the intensity of light passing through the portion of the ear positioned intermediate the light emitting and light receiving elements, and the sensor unit comprises means for outputting an electrical signal based thereon;
processor means, coupled to the sensor unit, for processing the electrical signal and determining a heart rate therefrom; and
a display, coupled to the processor means, for displaying the heart rate.

2. The heart rate monitor as claimed in claim 1, including a headset, to which the earpiece is coupled, wherein the display is mechanically coupled to the headset and provided on a visor.

3. The heart rate monitor as claimed in claim 1, comprising:
a headset to which the earpiece is coupled; and
an eyewear unit that is physically disconnectable from the headset;
wherein the eyewear unit is provided with the display for displaying the heart rate determined by the processor means.

4. The heart rate monitor as claimed in claim 1, including a wrist worn unit, wherein the wrist worn unit comprises the display for displaying the heart rate determined by the processor means.

5. The heart rate monitor as claimed in claim 4, including a headset with a transmitter coupled to the processor means, wherein the wrist worn unit wirelessly communicates with the transmitter.

6. The heart rate monitor as claimed in claim 1, including means for storing information relating to the heart rate.

7. The heart rate monitor as claimed in claim 6, including means for downloading the information to a stand-alone computer unit.

8. A system for playing back stored heart rate information, including a computer coupleable to the heart rate monitor as claimed in claim 7, wherein the computer comprises means for displaying the information downloaded from the means for storing information.

9. The heart rate monitor as claimed in claim 3, including a headset with a transmitter coupled to the processor means, wherein the transmitter wirelessly communicates heart rate information to the display.

10. A wearable fitness heart rate monitor comprising:
an earpiece comprising a sensor unit having a transmitting element and a receiving element, wherein the transmitting element and the receiving element are positioned adjacent each other, wherein the sensor unit is placed proximate to a portion of an ear, wherein the transmitting element emits light into the portion of the ear positioned proximate thereto and the receiving element receives reflected light therefrom, and wherein the sensor unit detects the intensity of the reflected light and outputs electrical signals based thereon;
processor means for processing the electrical signals and determining a heart rate therefrom; and
a display, coupled to the processor means, for displaying the heart rate.

11. The heart rate monitor as claimed in claim 10, comprising:
a headset to which the earpiece is coupled; and
an eyewear unit that is physically disconnectable from the headset;
wherein the eyewear unit is provided with the display for displaying the heart rate determined by the processor means.

12. The heart rate monitor as claimed in claim 10, including a wrist worn unit, wherein the wrist worn unit comprises the display for displaying the heart rate determined by the processor means.

13. The heart rate monitor as claimed in claim 12, including a headset with a transmitter coupled to the processor means, wherein the wrist worn unit wirelessly communicates with the transmitter.
